(19) United States
(12) Patent Application Publication
(10) Pub. No.: US 2023/0123450 A1
(43) Pub. Date: Apr. 20, 2023

(54) MEDICAL APPARATUS WITH EMBEDDED MARKING

(71) Applicant: OLYMPUS MEDICAL SYSTEMS CORP., Tokyo (JP)

(72) Inventors: Hiroaki KASAI, Akishima-shi (JP); Issei MAEDA, Sagamihara-shi (JP); Hirotaka KITAGAWA, Yokohama-shi (JP); Takashi MAGARA, Nishitokyo-shi (JP)

(73) Assignee: OLYMPUS MEDICAL SYSTEMS CORP., Tokyo (JP)

(21) Appl. No.: 17/886,596

(22) Filed: Aug. 12, 2022

Related U.S. Application Data
(60) Provisional application No. 63/252,210, filed on Oct. 5, 2021.

(51) Int. Cl. A61B 1/00; A61M 25/01

(52) U.S. Cl. CPC A61B 1/00101; A61M 25/0105; A61M 2025/0008

(57) ABSTRACT

A medical apparatus configured for insertion directly or indirectly into a living body is provided with a coloring part having a marking formed on an outer surface. The marking has a coloring layer of an acrylic resin containing a pigment and a protective layer of a polymethacrylic ester covering the coloring layer directly or indirectly, and an upper surface of the coloring part is recessed from a part of the medical apparatus, such as a covering tube of a sheath of an endoscope treatment instrument, adjacent to the coloring part on the outer surface.

### RELATED APPLICATION DATA

This application is based on and claims priority under 37 U.S.C. §119 to U.S. Provisional Application No. 63/252,210 filed on Oct. 5, 2021, the entire contents of which are incorporated herein by reference.

### FIELD OF THE INVENTION

The present invention relates to a medical apparatus, a marking and a marking forming method .

### DESCRIPTION OF THE RELATED ART

Heretofore, endoscopic treatment tools such as biopsy forceps have been used in endoscopic treatment. Endoscopic procedures such as biopsy forceps, in which a flexible sheath with a coated tube is inserted into the channel of the endoscope. A printed marking is provided on the outer peripheral surface of the covering tube, and the timing at which the treatment instrument protrudes from the tip of the endoscope and the timing at which the treatment instrument is pulled out from the endoscope are easily understood, thereby improving the efficiency of the procedure.

The marking requires durability to the extent that it does not fall off even when wiped with alcohol or the like, but in order to secure durability in printing, it is desirable that the ink used is of a thermosetting type, and it is difficult to suppress the cost. Patent Document 1 describes one in which a marker is printed in a recessed portion formed on an outer peripheral surface of a catheter. In this case, since the concave portion is formed to be large in a dish shape, there arises a problem that the printed marker is peeled off when the outer peripheral surface is wiped with a cloth unless the thermosetting ink is used and the ink itself has durability. Therefore, marking which is excellent in durability and can be reduced in cost has been desired.

Prior art documents - Patent Document 1: U.S. Pat. Publ. No. 2015/0148601.

### SUMMARY OF THE INVENTION

According to a first aspect of the present disclosure, a medical apparatus includes a marking formed on an outer surface, wherein the marking is provided with a coloring part having a coloring layer of an acrylic resin containing a pigment and a protective layer of a polymethacrylic ester covering the coloring layer directly or indirectly, and an upper surface of the coloring portion is recessed from a portion adjacent to the coloring portion on the outer surface. The medical apparatus is configured to be inserted directly or indirectly into a living body.

In the above medical apparatus, it is preferable that the width of the colored portion is 3 mm or less.

In the above medical apparatus, it is preferable that an upper surface of the colored portion is recessed by 5 µm or more relative to a portion adjacent to the colored portion on the outer surface.

In the medical apparatus, it is preferable that an inclination of an inner wall surface of a concave portion formed around the colored portion is 60 degrees or more with respect to the outer surface.

In the above medical apparatus, it is preferable that the pigment is titanium oxide particles.

In the above medical apparatus, it is preferable that the titanium oxide particles contained in the colored layer be 40 to 80 wt%.

In the medical apparatus described above, it is preferable that the marking is formed by a transfer process, such as with a transfer foil. The transfer process embeds the marking into a surface of a body of the medical apparatus, such as a covering tube of a sheath of an endoscope treatment instrument.

According to a second aspect of the present invention, a marking is formed in the medical apparatus to be inserted into an endoscope.

According to a third aspect of the present invention, a method of forming a marking on an outer surface of a medical apparatus which is inserted directly or indirectly into a living body comprises: a step of contacting a portion of a transfer foil to an outer surface of the medical apparatus, wherein the transfer foil is a multilayer including a colored layer of an acrylic resin containing a pigment, a colored portion having a protective layer of a polymethacrylic acid ester directly or indirectly covering the colored layer and a thin film base material made of a resin provided on the protective layer, wherein a first surface of the transfer foil on which the colored layer is formed is brought into contact with an outer surface of the medical apparatus; a step of heating a second surface of the transfer foil on which the thin film base material is provided and transferring the thin film base material by pressing the thin film base material toward the medical apparatus; and a step of separating the transfer foil from the medical apparatus.

In the method of forming the marking, it is preferable that the medical apparatus is formed in a cylindrical shape, the transfer foil is brought into contact with a part of the outer peripheral surface of the medical apparatus, and the medical apparatus is rotated about the cylindrical axis of the medical apparatus, whereby the contact portion of the transfer foil in contact with the outer peripheral surface of the medical apparatus is moved.

In the method of forming the marking, in the manufacturing process of transferring, it is preferable that the pressing member is retracted from the medical apparatus after the step of bringing the heated pressing member into contact with the second surface of the thin film base material, pressing the pressing member toward the medical apparatus to separate the transfer foil.

In the above method of forming the marking, it is preferable that the pigment is titanium oxide particles.

### BRIEF DESCRIPTION OF THE DRAWINGS

FIG. 1 is an overall view of an endoscope treatment system according to the first embodiment.

FIG. 2 is a general view showing a treatment instrument of the endoscope treatment system.

FIG. 3 is a side view of the distal end of the treatment instrument.

FIG. 4A is a schematic view in which a short treatment tool is provided in a short endoscope.

FIG. 4B is a schematic view of providing a long treatment tool in a long endoscope.

FIG. 4C is a schematic view in which a long treatment tool is provided in a short endoscope.

FIG. 5 is an enlarged side view of a sheath of a distal end portion of the treatment instrument.

FIG. 6 is a cross-sectional view of the sheath along line A-A shown in FIG. 5.

FIG. 7 is a cross-sectional view of the sheath along line B-B shown in FIG. 5.

FIG. 8 is a sectional view taken along line C-C shown in FIG. 7 is a sectional view taken along the marking from the circumferential direction.

FIG. 9 is a diagram showing a marking formation procedure.

FIG. 10 is a diagram showing a marking formation procedure.

FIG. 11 is a diagram showing a marking formation procedure.

FIG. 12 is a diagram showing a marking formation procedure.

FIG. 13 is a diagram showing a marking formation procedure.

FIG. 14 is a diagram showing a marking formation procedure.

FIG. 15 is a sectional view showing a marking according to the first modification.

FIG. 16 is a sectional view showing a marking according to a second modification.

FIG. 17 is a sectional view showing a marking according to a third modification.

FIG. 18 is a cross-sectional view showing a marking according to a fourth modification.

### DETAILED DESCRIPTION OF THE PREFERRED EMBODIMENT(S)

### Modes for Carrying Out the Invention

### First Embodiment

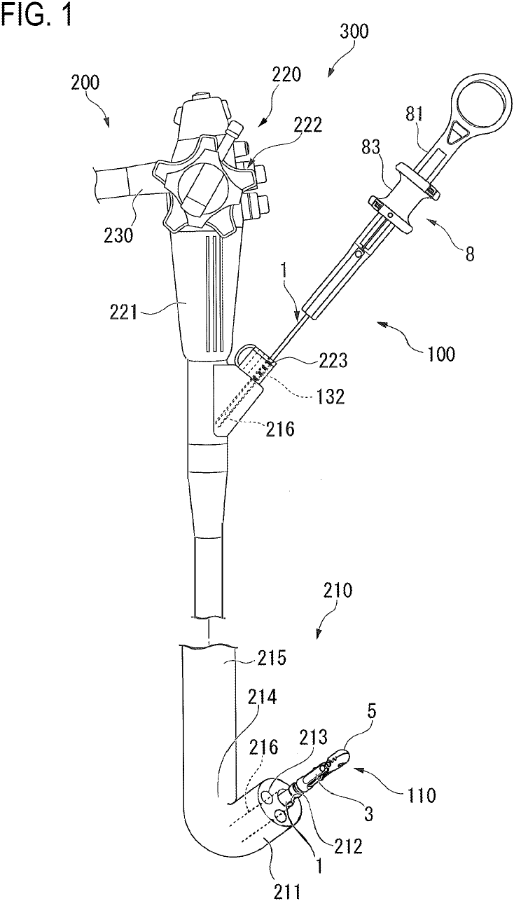

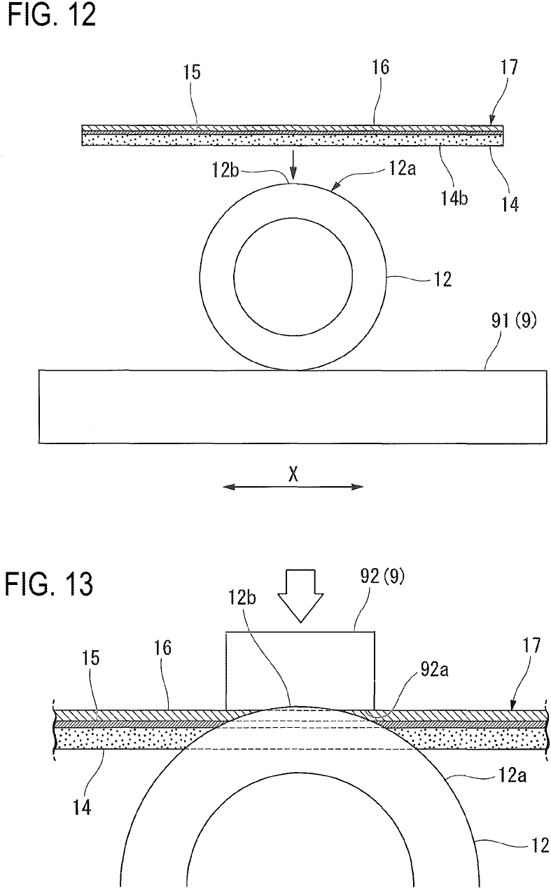

A medical apparatus, a marking and a method of forming the marking according to a first embodiment of the present invention will be described with reference to FIGS. 1 to 14. FIG. 1 is an overall view of an endoscopic treatment system 300.

### Endoscopic Treatment System 300

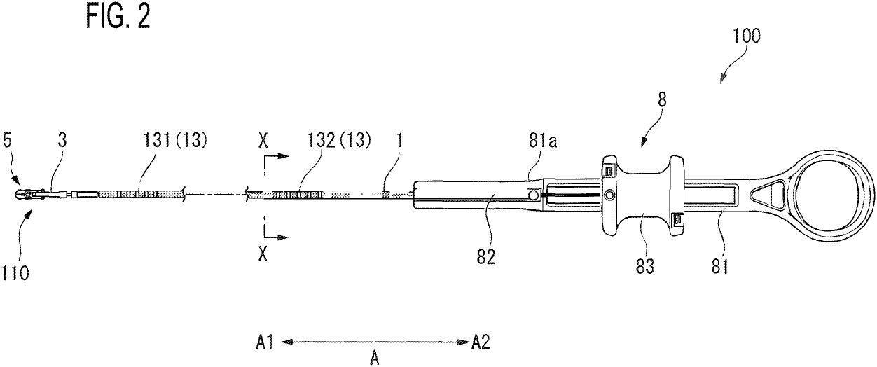

The endoscope treatment system 300 includes an endoscope treatment instrument 100 and an endoscope 200 as shown in FIG. 1. The endoscope treatment instrument 100 is used by being inserted into the endoscope 200. The endoscope treatment instrument 100 is a medical apparatus in which a marking 13 (see FIG. 2) is formed on an outer surface and inserted directly or indirectly into a living body.

### Endoscope 200

The endoscope 200 is a known flexible endoscope and includes an insertion portion 210 inserted into a body from a distal end, an operation unit 220 attached to a proximal end of the insertion portion 210, and a universal cord 230 attached to the operation portion 220.

The insertion portion 210 is an elongated member insertable into a body lumen. Insertion portion 210 has a distal end portion 211, a curvable portion 214, and a flexible portion 215, which are connected in this order from the distal end side. A channel 216 for inserting the treatment instrument 100 is provided inside the insertion portion 210. The distal end portion 211 also includes a distal end opening 212 and an imaging unit 213. The imaging unit 213 includes an image pickup device such as a CCD or a CMOS, for example, and can pick up an image of a region to be treated. The curvable portion 214 bends in accordance with the operation of the operation portion 220 by the user. The flexible portion 215 is a flexible tubular portion.

The operation unit 220 is connected to the flexible portion 215. The operation unit 220 includes a grip 221, an input unit 222, and an instrument port 223. The grip 221 is a member supported by a user. Input unit 222 receives an operation input for bending operation of the curvable portion 214. The instrument port 223 is a proximal opening of the channel 216.

The universal cord 230 connects the endoscope 200 and an external device. The universal cord 230 includes an imaging cable and an optical fiber cable or the like for outputting an imaging signal captured by the imaging unit 213 to the external device, such as a display.

### Endoscope Treatment Instrument 100

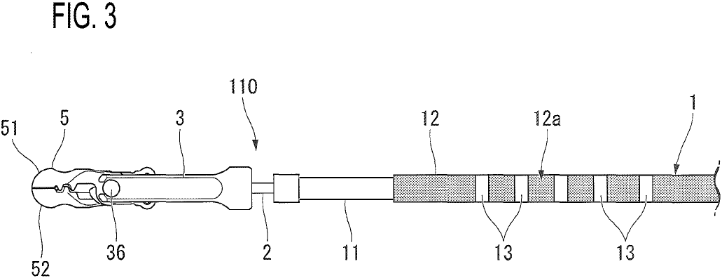

FIG. 2 is an overall view illustrating an endoscope treatment instrument 100. An endoscope treatment instrument 100 (also referred to as a treatment instrument 100) includes a sheath 1, an operation wire 2 (see FIG. 3), a support member 3, a procedure device such as forceps (jaw) 5, and an operation unit 8. In the following description, in the longitudinal direction A of the treatment instrument 100, the side to be inserted into the body of the patient is referred to as a “distal side A1”, and the side of the operation unit 8 is referred to as a “proximal side A2”. A support member 3 and a procedure device 5 are provided at a distal end portion of the treatment instrument 100. The procedure device 5 is rotatably supported by the support member 3. The support member 3 and the procedure device 5 constitute a “treatment portion 110” for treating an affected portion.

### Sheath 1

The sheath 1 is a flexible, elongate member extending from the distal end to the proximal end. The sheath 1 has an outer diameter that facilitates it being slidably inserted into the channel 216 of the endoscope 200. As shown in FIG. 1, when the sheath 1 is inserted into the channel 216, the distal end of the sheath 1 can protrude and retract from the distal opening 212 of the channel 216. The sheath 1 may have an insulating property.

FIG. 3 is an enlarged view of a main part of the treatment portion 110 shown in FIG. 2. The sheath 1 includes a coil sheath 11 formed by spirally winding a metal wire, a covering tube 12 covering the outer peripheral surface of the coil sheath 11, and a plurality of markings 13 provided on the outer peripheral surface 12a of the covering tube 12. The operation wire 2 is inserted in the inner space of the sheath 1.

### Marking 13

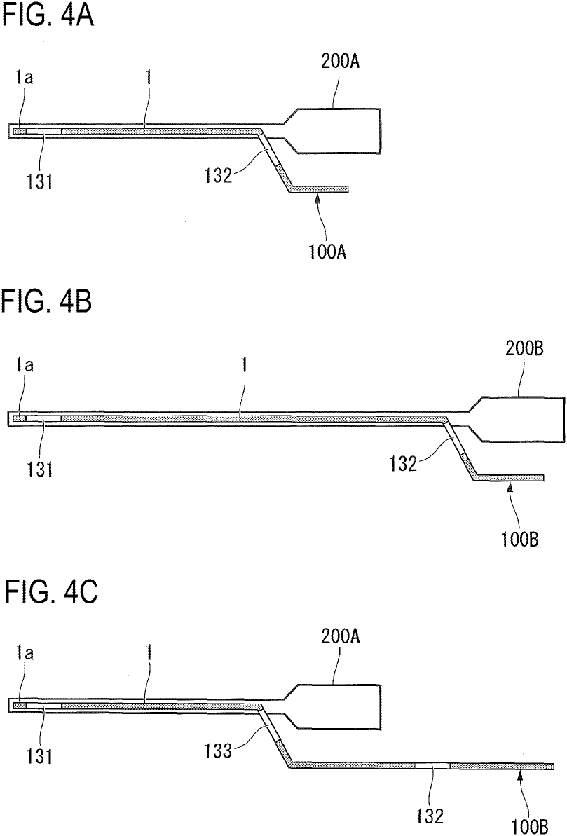

As shown in FIG. 2, the plurality of markings 13 are provided as a first marking 131 and a second marking 132. Each of the first marking 131 and the second marking 132 may be a single marking or a plurality of markings. FIG. 4A shows an illustration in which a short-length treatment instrument 100A is provided in a short-length endoscope 200A. As shown in FIG. 4A, the first marking 131 is provided near the distal end 1a of the sheath 1 and the second marking 132 is provided at a position where the second marking 132 is observable when the distal end of the treatment portion does not protrude from the short-length endoscope 200A. As shown in FIG. 4B, it is also possible to provide a long-length treatment instrument 100B in a long-length endoscope 200B. As shown in FIG. 4C, it is also possible to provide a long-length treatment instrument 100B in a short-length endoscope 200A. As shown in FIG. 4C, when the long-length treatment instrument 100B and the short-length endoscope 200A are combined, a third marking 133 may be provided on the sheath 1 of the long-length treatment instrument 100B at an intermediate position for combination of the long-length treatment instrument 100B and the short-length endoscope 200A. The third marking 133 may be a single marking or a plurality of markings.

The first marking 131 is provided at a position near the treatment portion 110 on the distal end side A1. When removing the treatment instrument 100 from the channel 216 of the endoscope 200, the user can recognize that the treatment portion 110 is approaching the instrument port 223 by checking the first marking 131. Therefore, the user can slowly withdraw the treatment portion 110, with which the living tissue or the like is grasped, from the instrument port 223 to remove the grasped living tissue from the treatment portion 110.

As shown in FIG. 1, when the treatment portion 110 protrudes from the distal end opening 212 of the endoscope 200 or just before the treatment portion 110 protrudes from the distal end opening 212 of the endoscope 200, the second marking 132 and, where applicable, the third marking 133 (see FIG. 4C) are provided at a position where the second marking 212 or the third marking 213 is about to be inserted into the channel 216 from the instrument port 223. When inserting the treatment instrument 100, the user can recognize the position of the treatment portion 110 with respect to the distal opening 212 by confirming the position of the second marking 132 (and, where applicable, the third marking 133) with respect to the instrument port 223. Therefore, the user can slowly project the treatment portion 110 from the distal end opening 212 of the endoscope 200, for example.

A specific configuration and a method of creating the first marking 131 and the second marking 132 will be described later.

### Operation Wire 2

Operation wire 2 is a metal wire and is inserted through the inner space of the sheath 1, e.g., a channel within the sheath 1. The distal end of the operation wire 2 is connected to the procedure device 5, and the proximal end of the operation wire 2 is connected to the operation section 8. The operation wire 2 includes, for example, two wires.

### Support Member 3

The support member 3 is attached to the distal end of the sheath 1 and supports the procedure device 5 as described below by a pin 36, which is a rotation axis. Central axis in the longitudinal direction A of the support member 3 substantially coincides with the central axis in the longitudinal direction A of the sheath 1. The inner space of the support member 3 communicates with the inner space of the sheath 1. Operation wire 2 inserts the inner space of the support member 3.

### Procedure Device (Jaws) 5

The procedure device, such as jaw 5, is a member for collecting biological tissue or performing a treatment, such as hemostatic coagulation. The procedure device 5 is formed of a metal material (such as stainless steel) and, in the case of a jaw, includes a pair of first forceps piece 51 and a second forceps piece 52. The first forceps piece 51 is rotatably supported by the pin 36 about the center axis of the pin 36. The first forceps piece 51 is operated by a first operation wire 21 (not shown) connected to the proximal end side A2 of the first forceps piece 51. The second forceps piece 52 is rotatably supported by the pin 36 about the center axis of the pin 36. The second forceps piece 52 is operated by a second operation wire 22 (not shown) connected to the proximal end side A2 of the second forceps piece 52. Note that one of the first forceps piece 51 and the second forceps piece 52 may be fixed to the support member 3, and only the other may be rotatably supported by the support member 3. The procedure device 5 may further have a linkage mechanism, and may be operated by a single operation wire connected to the linkage mechanism.

### Operation Unit 8

As shown in FIGS. 1 and 2, the operation unit 8 is provided on the proximal end side A2 of the sheath 1. The operation unit 8 includes an operation unit main body 81, a main body cover 82, and a slider 83.

The distal end portion 81a of the operation portion main body 81 is connected to the proximal end of the sheath 1. From the proximal end of the sheath 1, a pipe in which the operation wire 2 is inserted extends. The operation wire 2 and the pipe are fixed to the slider 83. The main body cover 82 is detachably attachable to the operation unit main body 81. A proximal end of the sheath 1 is fixed to the distal end portion 81 a of the operation unit main body 81 by mounting the main body cover 82 to the operation unit main body 81. The slider 83 is mounted so as to be movable along the longitudinal direction A with respect to the operation unit main body 81. The proximal end of the operation wire 2 is fixed to the slider 83 together with the pipe. When the user moves the slider 83 forward and backward relative to the operation unit main body 81, the operation wire 2 moves forward and backward.

Next, the markings 13 described above (131, 132, 133) will be specifically described. Since the first marking 131 and the second marking 132 shown in FIG. 2 have substantially the same configuration, the configuration of the markings 13 will be described below using the first marking 131.

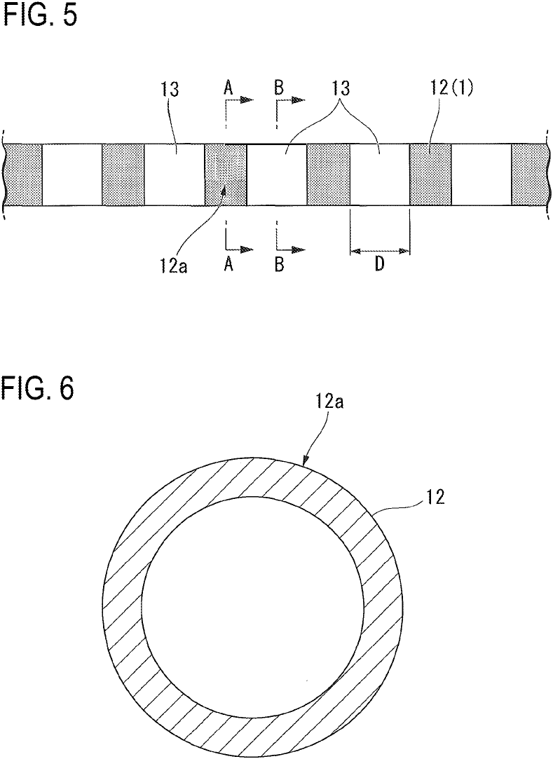

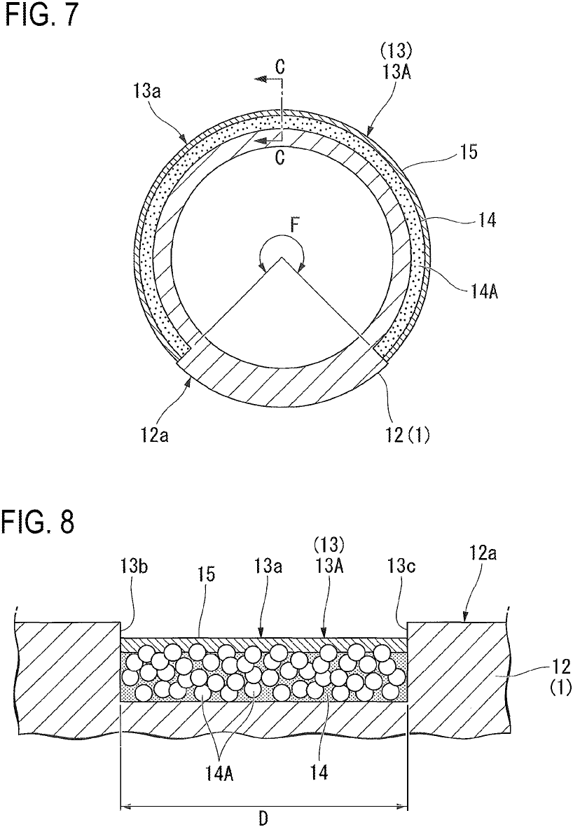

FIG. 5 is an enlarged view of the markings 13 shown in FIG. 3. FIG. 6 is a cross-sectional view taken along line A-A shown in FIG. 5 and FIG. 7 is a cross-sectional view taken along the line B-B shown in FIG. 5, and show cross-sectional views of the sheath 1 on which the markings 13 are formed. FIG. 8 is a sectional view taken along line C-C shown in FIG. 7, which shows a sectional view of a marking 13 from the circumferential direction. Incidentally, the scale of the thickness direction of the marking 13 is enlarged from the actual.

As shown in FIG. 5, a plurality of markings 13 are formed in a striped pattern at regular intervals along the longitudinal direction A of the treatment instrument 100. Marking 13 is formed along the circumferential direction of the sheath 1 with a constant width D (see also FIG. 7). Marking 13 is a region that extends in the circumferential direction by a predetermined amount (reference numeral F in FIG. 7). The marking 13 can extend in the circumferential direction to cover at least 75% of the circumference, i.e. is formed in region F, which is at least 75% of the circumference (270° region), or the marking 13 can extend in the circumferential direction to cover the entire circumference (360° region). As long as the marking 13 is formed in at least 75% in the circumferential direction (270° region), the marking 13 can be visually confirmed from any direction. Further, by providing the marking 13 just over 75% in the circumferential direction, it is possible to reduce the time required for forming the marking 13 and the material cost of the marking 13.

As shown in FIG. 8, the marking 13 is provided with a colored portion 13A having a colored layer 14 of an acrylic resin containing a pigment made of a titanium oxide particle 14A and a protective layer 15 of a polymethacrylic acid ester directly covering the colored layer 14. In particular, when a fluororesin component is mixed in the polymethacrylic ester protective layer, wiping resistance is further improved. In this embodiment, the protective layer 15 is directly laminated on the colored layer 14, but when a fluororesin containing protective layer is provided, for example, a protective layer 15 may be indirectly provided via a transparent adhesive layer such as an acrylic adhesive or an adhesive material having no clear interface such as a silane coupling agent.

By employing a titanium oxide particle 14A in the pigment of the coloring layer 14, a brilliant white marking 13 is obtained. In this embodiment, the titanium oxide particle 14A contained in the colored layer 14 is present in an amount of 40 wt% to 80 wt%. If the amount of the titanium oxide particles 14A is less than 40 wt%, the color becomes light and visibility decreases. In addition, when the amount of titanium oxide particles 14A exceeds 80 wt%, the particles exposed on the surface tend to come off easily, and the color bleeding occurs and the visibility is lowered.

The upper surface 13a of the colored portion of 13A is recessed by 5 µm or more from the portion of the outer peripheral surface 12a of the covering tube 12 of the sheath 1 adjoining to the colored portion 13A. That is, in the covering tube 12, a concave portion 13b is formed between the outer peripheral surface 12a and the upper surface 13a of the colored portion 13A and this concave portion 13b is recessed into the thickness of the body of the covering tube 12. In addition, when the amount of concavity of the concave portion 13b, e.g., the depth from the adjoining outer peripheral surface 12a of the covering tube 12, is less than 5 µm and the concavity is shallow, there is a possibility that the load becomes high when wiping the sheath 1. The upper limit of the amount of concavity of the concave portion 13b, e.g., the depth from the adjoining outer peripheral surface 12a of the covering tube 12, is preferably less than 50 µm for the reason that, if the amount of concavity is increased and the remaining sheath thickness becomes too thin, the flexibility decreases and bending becomes difficult.

The inner wall surface 13c of the concave portion 13b formed surrounding the colored portion 13A is perpendicular at 90 degrees with respect to the outer peripheral surface 12a of the covering tube 12. The angle of the inner wall surface 13c with respect to the outer peripheral surface 12a of the covering tube 12 is preferably set to 60 degrees or more.

The width D of the strip-shaped colored portions 13A of the markings 13 are set to 3 mm or less. By setting the width D to 3 mm or less, the colored portion 13A is hardly peeled off from the covering tube 12 when the sheath 1 is wiped with a cloth. However, when the width D exceeds 3 mm, the colored portion 13A is easily peeled off from the covering tube 12. In a product in which the number of wipes to be used over the lifetime is small, the width D may be 3 mm or more.

### Method of Forming Marking 13

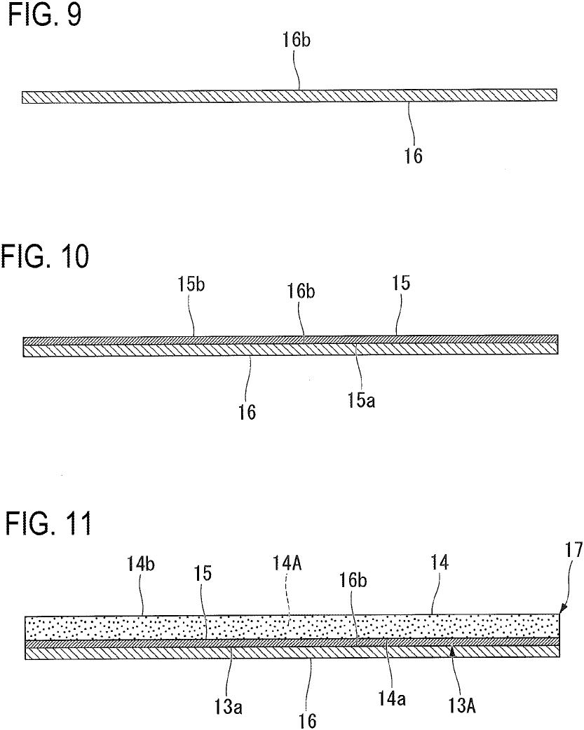

FIGS. 9 to 14 are diagrams showing a procedure for forming the marking 13. The marking 13 is formed by transfer using the transfer device 9 shown in FIG. 12. The method for forming the markings 13 includes a contacting step ST1, a transferring step ST2, and a separating step ST3.

First, as a preparation step, as shown in FIGS. 9 to 11, a multilayer is prepared to make a transfer foil 17 comprising a colored layer 14 of an acrylic resin containing a titanium oxide particle 14A, and a colored portion 13A having a protective layer 15 of a polymethacrylic ester directly covering the colored layer 14, and a sheet-like thin film substrate 16 made of a resin, such as polyethylene terephthalate (PET) provided on an upper surface 15a of the protective layer 15, for example. Specifically, as shown in FIGS. 9 and 10, with the back surface 16b of the thin film substrate 16 facing up, the protective layer 15 is applied in a detachable status by applying a polymethacrylic acid ester from above with respect to the back surface 16b thereof. Next, as shown in FIG. 11, with the back surface 15b of the protective layer 15 facing up, applying a colored layer 14 of acrylic resin from above with respect to the back surface 15b thereof. As a result, a colored portion 13A in which the surface 14a of the colored layer 14 is covered with the protective layer 15 is formed, and a transfer foil 17 in which a thin film substrate 16 is laminated on the upper surface 13a of the colored portion 13A is formed. When a fluorine-based resin is mixed in the protective layer 15, adhesion to the colored layer 14 is lowered, and therefore, it is also possible to provide between the protective layer 15 and the colored layer 14 an adhesive layer such as an acrylic adhesive or an adhesive material having no clear interface such as a silane coupling agent. In that case, between the application of the protective layer 15 and the colored layer 14, an adhesive layer and a coating step of an adhesive substance are provided.

Here, when transferring the transfer foil 17 to the covering tube 12 of interest for forming the marking 13, the transfer device 9 shown in FIGS. 12 to 14 is used. The transfer device 9 includes a stage 91 that is slidable in the lateral direction with the covering tube 12 placed thereon, moving means (not shown) that moves the transfer foil 17 so as to come into contact with the upper end portion 12b of the covering tube 12 placed on the stage 91 from above, and a pressing member 92 that heats at least the lower side 92a of the pressing member 92 and presses the portion of the transfer foil 17 that comes into contact with the upper end portion 12b of the covering tube 12 from above.

As shown in FIG. 14, the stage 91 is slidably provided in one direction (the sliding direction of reference numeral X in the paper plane lateral direction) by a driving means (not shown). The covering tube 12 is placed on the upper surface 91a of the stage 91 in the axial direction (longitudinal direction A of the treatment instrument 100) in a direction orthogonal to the sliding direction X in top view.

Pressing member 92 is heated at a predetermined temperature to the transfer foil 17 and is provided so as to be vertically movable. The pressing portion 92a of the bottom portion of the pressing member 92, the length of the direction perpendicular to the paper surface of FIG. 14 (the longitudinal direction A) is set to 3 mm or less which is the same dimension as the width D of the marking 13 (see FIG. 5). Further, the marking 13 formed in a plurality along the longitudinal direction A of the covering tube 12 may be realized by using the same number of independent pressing members 92 as markings 13, or using one pressing member 92 having the same number of pressing portions 92a as marking 13. That is, in the integral pressing member 92, a plurality of pressing portions 92a can be arranged in a comb-shape having a groove between the adjacent pressing portions 92a (when viewed from the sliding direction X), or is formed in a tooth shape.

### Contacting Process ST1

In the contacting step ST1 of the method of forming the marking, as shown in FIG. 12, the transfer foil 17 is reversed, and the back surface 14b on which the colored layer 14 is formed, i.e., the first surface, is brought into contact with the outer peripheral surface 12a of the cylindrical covering tube 12 from above. Specifically, the transfer foil 17 is moved downward by the moving means of the transfer device 9, and the downward movement is stopped at a position where the back surface 14b of the colored layer 14 contacts the uppermost portion of the outer peripheral surface 12a of the covering tube 12.

Incidentally, in the transfer foil 17, when the transfer foil 17 is created in a state in which the back surface 14b of the colored layer 14 is directed upward under the thin film substrate 16 in the procedure shown in FIGS. 9 to 11, the transfer foil 17 is vertically inverted in the contact step shown in FIG. 12.

### Transfer Process ST2

Next, as shown in FIG. 14, in the transfer process ST2, while heating the upper surface 16a (second surface) on which the thin film substrate 16 of the transfer foil 17 is provided, the thin film substrate 16 is transferred by pressing toward the outer peripheral surface 12a of the covering tube 12. Specifically, the heated pressing member 92 is moved downward to come into contact with the upper surface 16a of the thin film substrate 16, and the pressing member 92 is pressed toward the outer peripheral surface 12a of the covering tube 12. Then, the stage 91 slides in one of the sliding direction X (in FIG. 14 the paper surface right side X1) while in a state of the outer peripheral surface 12a of the covering tube 12 being pressed by the pressing member 92. Thus, while placed on the stage 91 in a state of the covering tube 12 being sandwiched by the stage 91 and the pressing member 92, the covering tube 12 is rotated around the cylindrical axis (counterclockwise direction E1 in FIG. 14) and moves relatively opposite to the sliding direction X (to paper surface left side X2 in FIG. 14) by rolling in response to the movement of the stage 91 in the sliding direction X. Since the contact portion of the transfer foil 17 in the pressing member 92 moves along with the rotation of the covering tube 12, the transfer foil 17 is transferred onto the outer peripheral surface 12a of the covering tube 12.

In the transfer process, the outer peripheral surface 12a is compressed by applying the heating and pressing by the pressing device 9 to the outer peripheral surface 12a of the covering tube 12 together with the transfer foil 17. As a result, the markings 13 formed on the outer peripheral surface 12a are formed in such a manner that the upper surface 13a of the colored portion 13A is recessed from the outer peripheral surface 12a adjoining the colored portion 13A by 5 µm or more as described above. Also, the marking 13 is melted into the surface of the covering tube 12 and the melting forms the concave portion 13b.

### Separation Process ST3

Next, in the separation process ST3, the thin film substrate 16 of the transfer foil 17 is separated from the outer peripheral surface 12a of the coating tubes 12. More specifically, when the transfer foil 17 is transferred to the area of the circumferential direction of the outer peripheral surface 12a of the covering tube 12, movement of the stage 91 in the X1 direction is stopped, and the pressing member 92 is retracted upward. Thereafter, the thin film substrate 16 of the transfer foil 17 is peeled away from the colored portion 13A to form markings 13 on the outer peripheral surface 12a of the coating tubes 12 as shown in FIGS. 5, 7, and 8.

As described above, according to the medical apparatus of the present embodiment, the plurality of markings 13 is provided with a colored portion 13A having a colored layer 14 of an acrylic resin containing a pigment and a protective layer 15 of a polymethacrylic ester directly or indirectly covering the colored layer 14. The upper surface of the colored portion 13A is recessed from the portion of the outer peripheral surface 12a adjoining the colored portion 13A. As a result, since the colored portion 13A of the markings 13 is a concave portion recessed from the outer peripheral surface 12a of the covering tube 12, which is a medical apparatus, the external force at the time of alcohol-wiping before and after the treatment is hardly applied to the colored portion 13A inside the concave portion. Further, since the upper surface 13a of the colored portion 13A is covered with the protective layer 15 made of polymethacrylic ester, it is possible to suppress the falling-off of the particles which are pigments (here, titanium oxide particles 14A), and to suppress the color fading of the colored portion 13A. As described above, in the present embodiment, the visibility can be maintained by improving the durability of the plurality of markings 13.

In addition, in this embodiment, since the colored layer 14 is directly or indirectly covered by the protective layer 15 of the polymethacrylic ester to form the colored portion 13A in a layer shape, the colored portion 13A can be created by the transfer foil 17. Therefore, since it becomes possible to form the plurality of markings 13 having the protective layer 15 at once (in one step) by forming the concave portion by the method of forming by transfer, it is possible to provide a higher resistance to wipes by the plurality of markings 13, and moreover, it is possible to form the plurality of markings 13 inexpensively. On the other hand, in the case of forming by printing, the colored portion is always convex with respect to the outer surface, and in the case of the laser marker, concavities and convexities are not formed, so that a separate concave forming process is required to form the concavities. Therefore, in order to form the concave portion, a method of forming the concave portion by transfer is desirable.

Further, in the present embodiment, since the colored portion 13A of the plurality of markings 13 forms a concave portion, the presence of the concave portion associated with the plurality of markings 13 can be recognized by manually touching the outer peripheral surface 12a of the covering tube 12 to confirm the condition of the plurality of markings 13.

Further, in the present embodiment, since the width D of the colored portion 13A is 3 mm or less, when the outer peripheral surface 12a of the covering tube 12 is wiped with a cloth, the colored portion 13A is hardly peeled off from the covering tube 12. When the width D of the colored portion 13A exceeds 3 mm, the wiping load on the colored portion 13A is increased, so that the colored portion 13A is more easily peeled off.

In addition, in the present embodiment, since the upper surface 13a of the colored portion 13A is recessed by 5 µm or more from the part of the outer peripheral surface 12a of the covering tube 12 that adjoins the colored portion 13A, it is possible to suppress the increase of loads when wiping the outer peripheral surface 12a of the covering tube 12. That is, in the case where the concave portion 13b formed with the plurality of markings 13 is less than 5 µm and the concave portion 13b is shallow, the load may be increased when wiping the sheath 1.

In the present embodiment, the inclination of the inner wall surface 13c of the concave portion 13b formed surrounding the upper surface 13a of the colored portion 13A is 60 degrees or more with respect to the outer peripheral surface 12a of the covering tube 12. If the angle is less than 60 degrees, the cross-section of the concave portion 13b becomes dish-shaped, and when the outer peripheral surface 12a is wiped, the cloth may touch the upper surface 13a of the colored portion 13A and scrape off the components.

In addition, in this embodiment, since the pigment constituting the coloring layer 14 is a titanium oxide particle 14A, it is possible to realize a marking 13 which becomes a thick and vivid white, which is conspicuous even in a dim endoscope chamber, for example, and can improve visibility.

In addition, in this embodiment, titanium oxide particles 14A are contained in the colored layer 14 in an amount of 40 to 80 wt%. By setting the content of the titanium oxide particle 14A in the coloring layer 14 in the range of 40 to 80 wt% as described above, the colored portion 13A is hardly peeled off, and the visibility can be further improved. In other words, in this embodiment, when the amount of titanium oxide particles 14A is less than 40 wt%, the color becomes thin and the visibility decreases, and when the amount of titanium oxide particles 14A exceeds 80 wt%, the particle density becomes large and titanium oxide particles 14A tend to be removed, and as a result, the color becomes thin and the visibility decreases.

In the present embodiment, the plurality of markings 13 is formed by transfer. In this case, since the colored portion 13A can be created by the transfer foil 17 and the plurality of markings 13 having the protective layer 15 can be formed at once by transfer, it is possible to provide to the plurality of markings 13 a higher resistance to wiping and to form the marking 13 at low cost.

In the present embodiment, the medical apparatus is formed in a cylindrical shape, the transfer foil is brought into contact with a part of the outer peripheral surface of the medical apparatus, and the medical apparatus is rotated about the cylindrical axis of the medical apparatus, whereby the contact portion of the transfer foil in contact with the outer peripheral surface of the medical apparatus is moved.

In the present embodiment, in the transferring process, the pressing member is retracted from the medical apparatus after the step of bringing the heated pressing member into contact with the second surface of the thin film base material, pressing the pressing member toward the medical apparatus to separate the transfer foil. As a result, the transfer foil can be efficiently transferred to the surface of the medical apparatus. Further, by using the pressing member, the outer peripheral surface 12a of the covering tube 12 is compressed by applying heat and pressure to the outer peripheral surface 12a together with the transfer foil 17 in the transfer process by pressing. As a result, the markings 13 formed on the outer peripheral surface 12a are efficiently formed such that the upper surface 13a of the colored portion 13A is recessed from the outer peripheral surface 12a adjoining the colored portion 13A.

According to the medical apparatus, the plurality of markings 13, and the method of forming the markings of the present embodiment, it is possible to form a marking which is excellent in resistance and can be reduced in cost.

Although the first embodiment of the present invention has been described above in detail with reference to the drawings, the specific configuration is not limited to this embodiment, and design changes and the like within a range not deviating from the gist of the present invention are also included. In addition, the constituent elements shown in the above-described embodiments and modified examples can be appropriately combined and configured.

### First Modification

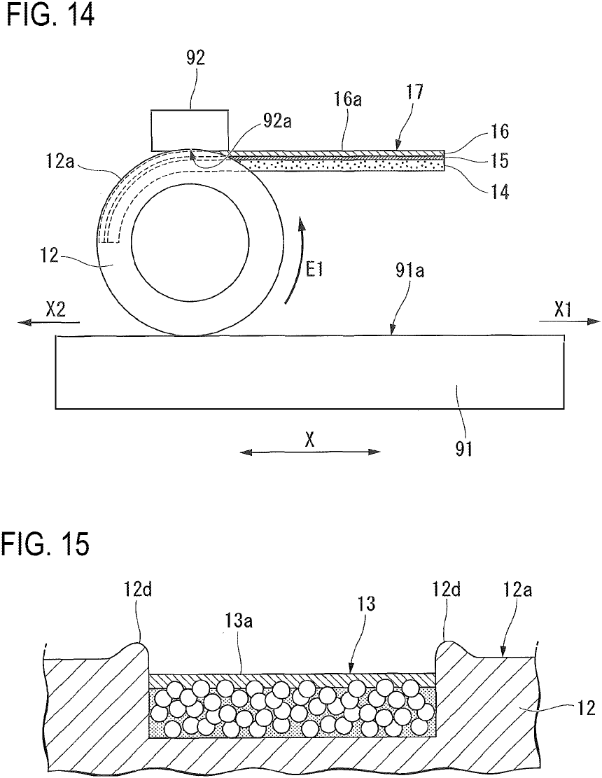

In the above-described embodiment, an example is shown in which, in the transfer process at the time of forming the marking 13, after heating and pressing the transfer foil 17, there is no change such that unevenness or the like occurs on the outer peripheral surface 12a of the covering tube 12 adjacent to the marking 13, but the present invention is not limited thereto. FIG. 15 is a sectional view showing a marking 13 according to the first modification. In the first modification shown in FIG. 15, the outer peripheral surface 12a of the covering tube 12 adjacent to the marking 13 has a raised upper portion 12d on the outside. The raised state of the raised upper portion 12d occurs due to pressing the transfer foil 17 with the pressing member 92 toward the outer peripheral surface 12a of the covering tube 12, as shown in FIG. 14 described above.

In the case of the first modification, the raised state can be confirmed by touching the outer peripheral surface 12a of the covering tube 12 around the marking 13 by hand. That is, by recognizing the presence of the raised portion 12d adjacent to the marking 13, it is possible to confirm that the marking 13 is formed by transfer.

### Second Modification

In the above embodiment, as shown in FIG. 8, the inner wall surface 13c of the concave portion 13b formed on the upper surface 13a of the colored portion 13A is perpendicular to the outer peripheral surface 12a of the covering tube 12 at 90 degrees. The angle of the inner wall surface 13c is preferably set to 60 degrees or more. FIG. 16 is a cross-sectional view showing a marking 13 according to a second modification. As shown in FIG. 16, in marking 13 according to the second modification, the angle 0 of the inner wall surface 13c is set to 60 degrees.

### Third Modification

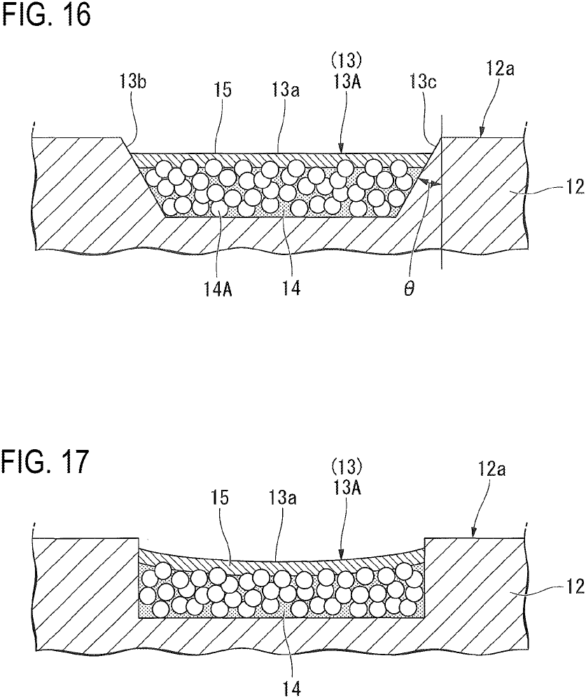

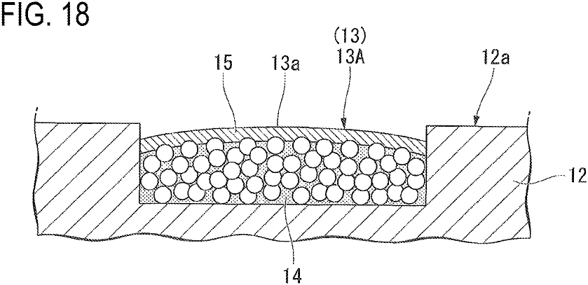

FIG. 17 is a cross-sectional view showing a marking 13 according to a third modification. As described above, the upper surface 13a of the marking 13 is not limited to the flat surface as in the above embodiment. In the third modification shown in FIG. 17, the upper surface 13a of the marking 13 is recessed in the radially inward direction (toward the center of the covering tube 12). This recessed upper surface 13a of the marking 13 can be formed, for example, by employing a convex shape of the pressing portion 92a of the pressing member 92 during the method of forming the marking.

In this way, the side portions of the marking 13 in the third modification are more closely aligned to the outer peripheral surface 12a of the covering tube 12 than in the above embodiment with a flat surface. Still, in the third modification the central region (in the width direction) of the upper surface 13a of the marking 13 is still further recessed from the outer peripheral surface 12a than both side portions. As a result, it is possible to more reliably suppress the cloth used when wiping from adhering or catching on the height transition from the marking 13 to the outer peripheral surface 12a of the covering tube 12. Still, since the central region of the upper surface 13a of the marking 13 can be positioned at a position deeper than the outer peripheral surface 12a, it is possible to prevent the marking from peeling off when wiped.

### Fourth Modification

FIG. 18 is a cross-sectional view showing a marking 13 according to the fourth modification. As described above, the upper surface 13a of the marking 13 is not limited to the flat surface as in the above embodiment. In the fourth modification shown in FIG. 18, the upper surface 13a of the marking 13 has a convex, radially outward shape. This recessed upper surface 13a of the marking 13 can be formed, for example, by employing a concave shape of the pressing portion 92a of the pressing member 92 during the method of forming the marking.

In this way the fourth modification, the central region (in the width direction) of the upper surface 13a of the marking 13 is more closely aligned with the outer peripheral surface 12a of the covering tube 12 than the side portions of the marking 13. As a result, it is possible to increase the visibility of the marking 13.

### Examples

Next, examples of marking corresponding to the above-described embodiments will be described together with comparative examples. The following Table 1, Table 2, and Table 3 show the schematic configurations and evaluation results of Examples 1 to 9 and Comparative Examples 1 to 4.

TABLE 1

| | | | | | |
|---|---|---|---|---|---|
| | Ex. 1 | Ex, 2 | Comp. Ex. 1 | Comp. Ex. 2 | Comp. Ex. 3 |
| protective layer of polymethacrylic acid ester | Not applied | Not applied | Not applied | Not applied | Not applied |
| amount of titanium oxide (wt%) | 40 | 60 | 30 | 40 | 60 |
| step (gap between a surface of a marking and a surrounding surface of a medical apparatus) (µm) | 5 | 5 | 5 | 1 | 5 |
| width of a marking (mm) | 3 | 3 | 3 | 3 | 5 |
| visibility | A | A | B | A | A |
| durable number of alcohol wiping | 10 or more B | 10 or more B | not tested | less than 10 C | less than 10 C |

TABLE 2

| | | | | | | | | |
|---|---|---|---|---|---|---|---|---|
| | Ex. 3 | Ex. 4 | Ex. 5 | Ex. 6 | Ex. 7 | Ex. 8 | Ex. 9 | Comp. Ex. 4 |
| protective layer of polymethacrylic acid ester | Applied | Applied | Applied | Applied | Applied | Applied | Applied | Applied |
| durability to alcohol wiping | 80 | 80 | 80 | 40 | 60 | 40 | 60 | 90 |
| step (gap between a surface of a marking and a surrounding surface of a medical apparatus) (µm) | 1 | 5 | 5 | 1 | 5 | 5 | 5 | 5 |
| width of a marking (mm) | 3 | 5 | 3 | 3 | 5 | 3 | 3 | 3 |
| visibility | A | A | A | A | A | A | A | A |
| durable number of alcohol wiping | 10 or more B | 10 or more B | 20 or more B | 20 or more B | 20 or more B | 30 or more A | 30 or more A | less than 10 C |

TABLE 3

| amount of titanium oxide (wt%) | | | 30 | 40 | 60 | 80 | 90 |
|---|---|---|---|---|---|---|---|
| visibility | | | B | A | A | A | A |
| durability to alcohol wiping | no protective layer | step 1 µm , width 3 mm | --- | Comp. Ex. 2 C | --- | --- | |
| | | step 5 µm, width 5 mm | --- | --- | Comp. Ex. 3 C | --- | |
| | | step 5 µm, width 3 mm | Comp. Ex. 1 B | Ex. 1 10 times B | Ex. 2 10 times B | --- | |
| | protective layer provided | step 1 µm , width 3 mm | | Ex.6 20 times B | - | Ex. 3 10 times B | |
| | | step 5 µm, width 5 mm | --- | | Ex. 7 20 times B | Ex. 4 10 times B | |
| | | step 5 µm, width 3 mm | | Ex. 8 30 times A | Ex. 9 30 times A | Ex. 5 20 times B | Comp. Ex. 4 C |

The markings of Examples 1 and 2 and Comparative Examples 1 to 3 shown in Table 1 do not have a protective layer of polymethacrylic ester. Examples 3 to 9 and Comparative Example 4 shown in Table 2 have a protective layer of a polymethacrylic ester. In the colored layer, an acrylic resin containing titanium oxide particles which display white color in the same manner as in the above embodiment is adopted.

Tables 1 and 2 report, for Examples 1 to 9 and Comparative Examples 1 to 4, the amount to titanium oxide content (wt%), the height (µm) of the step (i.e., the gap between the surface of the medical apparatus surface (corresponding to the outer peripheral surface 12a of the covering tube 12 of the above embodiment) and the surface of the marking (corresponding to the upper surface 13a of the marking 13 of the above embodiment), which corresponds to the recessed amount of the recess 13b of the above embodiment)), the line width (mm) of the marking (corresponding to the width D of the marking 13 of the above embodiment), the initial visibility, and the durability of the marking to alcohol wiping (reported in number of wipings). By this measure, the resistance of the marking 13 was evaluated and confirmed. Table 3 summarizes the marking conditions and results shown in Tables 1 and 2.

### Evaluation Method

The initial visibility is to confirm the visibility of the marking formed under the conditions shown in Tables 1 and 2 by visual inspection in a state before use, that is, a state before the alcohol wiping test is performed.

Visibility of Examples 1 to 9 and Comparative Examples 1 to 4 was envaulted visually. In the visibility evaluation, two grades are adopted. The state in which the white color of the marking was not significantly changed was evaluated as “good” (indicated by “A” in Table 1, Table 2, and Table 3), and the state in which the white color of the marking faded and the outer peripheral surface of the coating tube was whitish and bled as a whole was evaluated as “bad” (indicated by “B” in Table 1, Table 2, and Table 3). More specifically, the blurred state in the case of “defective” is a state in which the boundary between the printing portion and the non-printed portion becomes unclear and visibility is lowered. In Examples 1 to 9 and Comparative Examples 1 to 4, the number of times of alcohol wiping resistance was tested by performing, a plurality of times, wiping with a cloth containing alcohol on the marking to test the number of times that good visibility was maintained based on the above visibility evaluation method.

Alcohol-wiping resistance was evaluated in three grades as follows: “Very good” (indicated by “A” in Table 1, Table 2, and Table 3) when the alcohol-wiping was performed 30 times (i.e., 30 wipings) and the white color of the marking was clear without color fading; “good” (indicated by “B” in Table 1, Table 2, and Table 3) when the white color of the marking was clear, but had some fading visible to the naked eye during 10 to 29 alcohol-wipings; and “bad” (indicated by “C” in Table 1, Table 2, and Table 3) when the white color of the marking was not clear because of color fading within the first 10 alcohol-wipings.

### Evaluation Results

Examples 1 to 9 and Comparative Examples 1 to 4 were evaluated on the basis of the results shown in Table 1, Table 2, and Table 3.

In Examples 1 and 2, although the protective layer was not provided, since the titanium oxide content was 40 to 60 wt%, the step was 5 µm or more, and the line width was 3 mm or less, the load on the marking at the time of alcohol wiping was reduced. In Examples 1 and 2, it was found that color loss, i.e., fading, did not occur when the number of wipings was more than 10, and the evaluation was “B.”

In Comparative Example 1, since the content of titanium oxide was small, the color of the base material became translucent, and a good visibility could not be obtained.

In Comparative Examples 2 and 3, the titanium oxide content is 40 and 60 wt%, respectively, and the step or width were varied (relative to Examples 1 and 2) as indicated in Table 1. In Comparative Example 2, the step was 1 µm (i.e. was reduced), and it was found that color fading occurred when the number of wipings was less than 10, and the evaluation was “C.” In Comparative Example 3, the line width was 5 mm (i.e., was increased) and it was found that color fading occurred when the number of wipings was less than 10, and the evaluation was “C.”

In Examples 3 and 4, since it had a protective layer, it was found that color loss did not occur when the number of wipings was more than 10 (evaluated as “B”), even if the titanium oxide content was as large as 80 wt%

In Example 5, the protective layer was provided, a titanium oxide content was 80wt%, the step was 5 µm, and the line width was 3 mm, and it was found that color loss did not occur when the number of wipings was more than 20 times, and the evaluation was “B.”

In Examples 6 and 7, a protective layer was provided, a titanium oxide content was 40 or 60 wt%, respectively, and the step and line width were smaller in Example 6 (step of 1 µm and width of 3 mm) as compared to Example 7 (step of 5 µm and width of 5 mm). In both Examples 6 and 7, it was found that color loss did not occur when the number of wipings was more than 20 times, and the evaluation was “B.”

In Examples 8 and 9, a protective layer was provided, a titanium oxide content was 40 or 60 wt%, respectively, and the step and line width were the same (step of 5 µm and width of 3 mm), which is a smaller width as compared to Example 7 (step of 5 µm and width of 5 mm). In both Examples 6 and 7, it was found that color loss did not occur when the number of wipings was more than 30 times, and the evaluation was “A.”

In Comparative Example 4, it was confirmed that, even if the protective layer was provided, the titanium oxide content of 90 wt% was too large, and the particles protruded to the outer surface. For Comparative Example 4, it was found that color loss occurred when the number of wipings was more less than 10 times, and the evaluation was “C.”

The preferred embodiment and each modification of the present invention have been described above together with each example, but the present invention is not limited to these embodiments, each modification, and each example. Addition, omission, substitution, and other changes in configuration are possible without departing from the spirit of the present invention.

Also, the invention is not limited by the foregoing description, but only by the appended claims.

As the medical apparatus on which the marking is formed, not only those inserted into the medical apparatus (endoscope) and used in the living body but also those directly inserted into the living body are applicable.

Further, in the present embodiment, the cylindrical covering tube 12 is exemplified as the medical apparatus on which the marking is formed, but the cross-sectional shape of the medical apparatus is not limited to a circular shape, and other cross-sectional shapes such as a rectangular shape and an elliptical shape can be adopted.

In addition, in the present embodiment, a titanium oxide particulate 14A which becomes white is employed as a pigment, but is not limited to being white. For example, it is also possible to use a colored layer of an acrylic resin mixed with black titanium oxide as a black or gray color.

In the embodiment described above, a linear mark extending in the circumferential direction on the surface of the cylindrical medical apparatus is employed as the marking 13, but the marking is not limited to such a mark. For example, markings such as numbers, letters, and symbols may be formed by transfer or the like.

Further, as the thin film substrate 16 of the transfer foil 17, a sheet-like material made of resin such as polyethylene terephthalate (PET) is employed, but it is not limited to PET, and for example, PP, PE, or the like can be used, but it is desirable to use PET because of its high heat resistance.

In the above embodiment, the endoscopic treatment instrument is a biopsy forceps, but the endoscopic treatment instrument is not limited thereto. The endoscope treatment instrument may be, for example, a grasping forceps having a pair of forceps pieces similarly or a hemostatic forceps objective for hemostasis coagulation.

### Industrial Applicability

The present invention can be applied to medical apparatus, markings per se, and methods of forming markings.

### Description of Symbols

300 ENDOSCOPIC PROCEDURE SYSTEM

200 Endoscope

100 Treatment tools (endoscopic instruments)

1 Sheath

12 Covering tube

12a Outer peripheral surface

13 Marking

13A Colored portion

14 Colored layer

15 Protective layer

16 Thin film substrate

17 Transfer foil

2 Operating wire

3 Support member

4 Needle member

5 Forceps (jaws)

8 Operation Unit

9 Transfer apparatus

91 Stage

92 Pressing member

What is claimed is:

1. A medical apparatus configured to be inserted directly or indirectly into a living body, the medical apparatus comprising:
a plurality of markings formed on an outer surface of the medical apparatus,
wherein at least one of the plurality of markings include a colored portion that includes a colored layer comprising acrylic resin and a pigment and a protective layer comprising polymethacrylic acid ester,
wherein the protective layer covers the colored layer directly or indirectly, and
wherein an upper surface of the colored portion is recessed from an outer peripheral surface of a portion of the medical apparatus adjoining to the colored portion.

2. The medical apparatus according to claim 1, wherein a width of the colored portion in a circumferential direction of the medical apparatus is 3 mm or less.

3. The medical apparatus according to claim 1, wherein a depth of the upper surface of the colored portion recessed from the outer peripheral surface of the portion of the medical apparatus adjoining to the colored portion is 5 µm or more.

4. The medical apparatus according to claim 1, wherein the upper surface of the colored portion has a concave shape and defines a concave portion, and wherein an inner wall surface of the concave portion is oriented with respect to the outer peripheral surface at an angle of 60 degrees or more.

5. The medical apparatus according to claim 1, wherein the pigment is a titanium oxide particle.

6. The medical apparatus according to claim 5, wherein an amount of the titanium oxide particles contained in the colored layer is 40-80 wt%.

7. The medical apparatus according to claim 1, wherein a width of the colored portion in a circumferential direction of the medical apparatus is 3 mm or less,
wherein a depth of the upper surface of the colored portion recessed from the outer peripheral surface of the portion of the medical apparatus adjoining to the colored portion is 5 µm or more,
wherein the upper surface of the colored portion has a concave shape and defines a concave portion, and wherein an inner wall surface of the concave portion is oriented with respect to the outer peripheral surface at an angle of 60 degrees or more,
wherein the pigment is a titanium oxide particle, and
wherein an amount of the titanium oxide particles contained in the colored layer is 40-80 wt%.

8. The medical apparatus according to claim 7, wherein the medical apparatus is a covering tube of a sheath of an endoscope treatment instrument.

9. The medical apparatus according to claim 1, wherein the plurality of markings include a first plurality of markings located at a distal end region of the medical apparatus and a second plurality of markings located at a proximal end region of the medical apparatus, and
wherein both the first plurality of markings and the second plurality of markings include one of the at least one of the plurality of markings that include the colored portion.

10. The medical apparatus according to claim 9, wherein, in each of the first plurality of markings and the second plurality of markings, the one of the at least one of the plurality of markings that includes the colored portion has:
a width of the colored portion in a circumferential direction of the medical apparatus that is 3 mm or less,
a depth of the upper surface of the colored portion recessed from the outer peripheral surface of the portion of the medical apparatus adjoining to the colored portion that is 5 µm or more,
a concave shape of the upper surface of the colored portion that defines a concave portion, wherein an inner wall surface of the concave portion is oriented with respect to the outer peripheral surface at an angle of 60 degrees or more, and
an amount of pigment contained in the colored layer that is 40-80 wt%, where the pigment is a titanium oxide particle.

11. The medical apparatus according to claim 10, wherein the medical apparatus is a covering tube of a sheath of an endoscope treatment instrument.

12. The medical apparatus according to claim 1, wherein the plurality of markings is formed by a transfer process.

13. The medical apparatus to claim 1, wherein the medical apparatus has a cylindrical shape, and wherein the plurality of markings extends in a circumferential direction of the medical apparatus to cover at least 75% of a circumference of the medical apparatus.

14. A marking formed on the medical apparatus according to claim 1.

15. A method of forming a marking on an outer surface of a medical apparatus configured to be inserted into a living body, the method of forming the marking comprising:
contacting a portion of a transfer foil to an outer surface of the medical apparatus, wherein the transfer foil is a multilayer including a colored layer of an acrylic resin containing a pigment, a protective layer of a polymethacrylic acid ester directly or indirectly covering the colored layer, and a thin film substrate made of resin provided on the protective layer, and wherein the portion of the transfer foil contacting the outer surface of the medical apparatus is the colored layer;
transferring the multilayer to the outer surface of the medical apparatus by heating the multilayer while pressing the multilayer toward the medical apparatus; and
removing the thin film substrate from the medical apparatus,
wherein, during transferring, the marking is formed on the outer peripheral surface,
wherein the marking includes the colored layer and the protective layer, the colored layer and the protective layer defining a colored portion of the marking, and
wherein an upper surface of the colored portion is recessed from an outer peripheral surface of a portion of the medical apparatus adjoining to the colored portion.

16. The method of forming a marking according to claim 15, wherein the medical apparatus is formed in a cylindrical shape,
wherein contacting the transfer foil to the outer surface of the medical apparatus contacts the transfer foil with a part of an outer peripheral surface of the medical apparatus, and
wherein, during transferring, the medical apparatus is rotated about a cylindrical axis of the medical apparatus and the portion of the transfer foil contacting the outer surface of the medical apparatus moves.

17. The method of forming a marking according to claim 16, wherein transferring includes contacting a heated pressing member with a surface of the thin film substrate, and pressing the heated pressing member toward the medical apparatus, and
wherein, after completion of transferring, the pressing member is retracted from the medical apparatus.

18. The method of forming a marking according to claim 15, wherein the pigment is a titanium oxide particle.

19. The method of forming a marking according to claim 15, wherein the medical apparatus is formed in a cylindrical shape,
wherein a width of the colored portion of the marking in a circumferential direction of the medical apparatus is 3 mm or less,
wherein a depth of the recessed upper surface of the colored portion from the outer peripheral surface of the portion of the medical apparatus adjoining to the colored portion is 5 µm or more,
wherein the upper surface of the colored portion has a concave shape and defines a concave portion,
wherein an inner wall surface of the concave portion is oriented with respect to the outer peripheral surface at an angle of 60 degrees or more, and
wherein an amount of pigment contained in the colored layer is 40-80 wt%, where the pigment is a titanium oxide particle.

20. The method of forming a marking according to claim 19, wherein the medical apparatus is a covering tube of a sheath of an endoscope treatment instrument.

21. A marking formed on the medical apparatus by the method of forming a marking according to claim 15.
